(19) United States
(12) Patent Application Publication
(10) Pub. No.: US 2023/0191074 A1
(43) Pub. Date: Jun. 22, 2023

(54) Handheld Relaxation Device

(71) Applicant: Nelson Diaz Martinez, Orlando, FL (US)

(72) Inventor: Nelson Diaz Martinez, Orlando, FL (US)

(21) Appl. No.: 18/067,077

(22) Filed: Dec. 16, 2022

Related U.S. Application Data
(60) Provisional application No. 63/292,361, filed on Dec. 21, 2021.

(51) Int. Cl. A61M 21/02; A63H 33/00

(52) U.S. Cl. CPC A61M 21/02; A63H 33/00; A61M 2021/0022

(57) ABSTRACT

A handheld relaxation device includes a nut and bolt that is configured to fit in a user's hand and help relieve stress in adults and children.

### CROSS-REFERENCE TO RELATED APPLICATIONS

This application claims the benefit of U.S. Provisional Application No. 63/292,361 filed on Dec. 21, 2021, which is hereby incorporated in its entirety.

### FIELD OF THE INVENTION

The present invention is directed to a handheld relaxation device. In particular, the invention is directed to a nut and bolt that is configured to fit in a user's hand to help relieve stress in adults and children.

### BACKGROUND OF THE INVENTION

Many people suffer from anxiety, attention deficit hyperactivity disorder (ADHD), attention deficit disorder (ADD), hyperactivity, autism spectrum disorder (ASD), or even experience concentration issues from time to time. One of the common symptoms in these types of concentration disorders is restlessness or nervousness which can cause a person to fidget or move subconsciously. Furthermore, restlessness or fidgeting can occur in a person who has not been diagnosed with one of these concentration disorders, i.e., someone who is nervous about an upcoming presentation in school or at work.

In a group setting, it can be distracting to others in the group when someone is restless, fidgeting, or cannot stop moving. The restless person needs a way to expel this nervous energy so as not to distract themselves or others. It is common knowledge that performing a physical movement (tapping foot) or using a physical object (fidget toy), when the person is anxious or nervous, can help calm their mind and enable them to focus on their task at hand. Michael Hinck, Can Fidget Toys Help Your Child's Ability to Focus?, Flushing Hospital, Apr. 13, 2017, https://www.flushinghospital.org/newsletter/can-fidget-toys-help-your-childs-ability-to-focus/

There are different fidget toys and stress relieving balls, but none that combine a fidget and/or relaxation device with music. For example, the Bolt Nut Stress Toy is in the shape of a nut and bolt, however, this device is made of a squeezable foam and the nut cannot be removed from the bolt. https://www.qualitylogoproducts.com/stress-balls/bolt-nut-stress-toy.htm

One of the symptoms of prolonged computer use is pain and/or stiffness in the shoulders, arms, and hands. This affects individuals who are on the computer for long periods of time, e.g., gamers, computer programmers, data entry professionals, college professors, etc. One of the ways to relieve the stress on the shoulders, arms, and hands is to move the hands (by using a stress ball or fidget device) from the tension filled position of typing or using the mouse enabling increased blood flow to the hands.

Therefore, there exists a need for a relaxation device that can used in connection with music to enhance the relaxation experience and reduce stress and anxiety.

### SUMMARY OF THE INVENTION

The present invention is directed to a handheld relaxation device directed to self-regulate stress and anxiety in individuals. The device is configured to expel nervous energy, relieve stress, and increase blood circulation by generating a rotational movement with the user's hands. In another embodiment, the device is used as a meditation tool. In yet another embodiment, the device is configured to strengthen hand muscles and prevent “computer claw.” The handheld relaxation device can be used in combination with lo-fi music to increase relaxation while using the device. The device is constructed in different sizes, and it is configured to fit in the palm of the user's hand. In particular, the device can be effective at helping with focus and concentration in persons with anxiety, ADHD, ADD, hyperactivity, ASD, or who experience concentration issues.

The handheld relaxation device is configured to be used while listening to lo-fi music. Lo-fi music is audio that is of low-quality or low fidelity. It has components such as background noises, static, and/or repetitive loops. Listening to lo-fi music has been known to increase productivity, heighten brain activity to increase concentration, and release dopamine to help elevate an individual's mood. https://promusicianhub.com/what-is-lo-fi-music/ Listening to lo-fi music while spinning the nut onto the bolt of the handheld relaxation device will increase concentration in the user and help reduce their stress and anxiety levels.

The handheld relaxation device is configured to relieve stress, calm anxiety, and aid in motor skills therapy for the hands through the continuous movement of screwing and unscrewing the nut on to the bolt. This device is small, discrete, and easy to hold in one hand. In addition, the handheld relaxation device can help people who have difficulty focusing or even gamers who spend many consecutive hours with a hand controller. The users' hands will have increased circulation by holding the bolt portion of the device in one hand and repetitively screwing and unscrewing the nut portion of the device with the opposite hand.

In an exemplary embodiment, the handheld relaxation device comprises (a) a bolt, wherein the bolt comprises a bolt head and bolt threads; (b) a nut, wherein the nut comprises a plurality of nut side and a nut center; (c) wherein the nut is removably attached to the bolt by rotating the nut center to engage the bolt threads of the bolt; wherein the bolt threads readily engage threads in the nut center. In one embodiment, the bolt head and the nut are in the shape of a hexagon.

The material of construction for nut and bolt of the handheld relaxation device comprises acrylonitrile butadiene styrene (ABS), nylon, glycol modified polyethylene terephthalate (PETG), polycarbonate, polyvinyl alcohol (PVA), acrylic styrene acrylonitrile (ASA), or any combination thereof.

In one embodiment, the method of manufacture for the device comprises 3D printing. In an alternate embodiment, the method of manufacture for the device comprises injection molding.

In an alternate embodiment, the bolt head further comprises a plurality of bolt head grooves, a plurality of bolt head notches, a bolt head top and a bolt head bottom. In an embodiment, the nut further comprises a plurality of nut grooves, plurality of nut notches, a nut top, and a nut bottom.

In yet another embodiment, the circumference of the bolt head top is smaller than the circumference of the bolt head bottom; the bolt head bottom tapers down to the bolt head top; the circumference of the nut top is smaller than the circumference of the nut bottom, and the nut bottom tapers down to the nut top.

In another embodiment, the circumference of the bolt head top is equal to the circumference of the bolt head bottom; the bolt head bottom and the bolt head top taper towards the middle of the bolt head; the circumference of the nut top is equal to the circumference of the nut bottom, and the nut bottom and the nut top taper towards the middle of the nut.

In one embodiment, the bolt head and the nut comprise a plurality of swirls. In yet another embodiment, the bolt head and the nut are of equal size.

In an exemplary embodiment, the bolt is about 0.67″ to 3.44″ in width, about 0.67″ to 3.44″ in length, and about 1.51″ to 6.25″ in height, and the nut is about 0.67″ to 3.44″ in width and about 0.67″ to 3.44″ in length.

In yet another embodiment, the handheld relaxation device comprises (a) a bolt, wherein the bolt comprises a bolt head, wherein bolt head comprises a plurality of bolt grooves and bolt notches and bolt threads; (b) a nut, wherein the nut comprises a plurality of nut side, a nut center, a plurality of nut grooves, and a plurality of nut notches; (c) a cap, wherein the cap comprises a cap center; wherein the nut is removably attached to the bolt by rotating the nut center to engage the bolt threads of the bolt; wherein the bolt threads readily engage threads in the nut center; and wherein the cap is removably attached to the bolt by rotating the cap center to engage the bolt threads of the bolt; wherein the bolt threads readily engage threads in the cap center.

In this embodiment, the bolt is about 2.91″ to 3.44″ in width, about 2.91″ to 3.44″ in length, and about 4.53″ to 6.25″ in height; the nut is about 2.91″ to 3.44″ in width and about 2.91″ to 3.44″ in length; and the cap is about 2.91″ to 3.44″ in width and is about 2.91″ to 3.44″ in length.

A preferred method of use of a handheld relaxation device comprises: (a) holding the bolt in a user's hand with bolt head cradled in the palm of the hand and fingertips placed in several of the plurality of bolt head grooves; (b) holding the nut in the user's opposite hand with the users' fingertips in several of the plurality of nut grooves; (c) removably attaching the nut to the bolt by threading the nut center onto the bolt threads; and (d) removably detaching the nut from the bolt by unscrewing the nut from the bolt threads.

An alternate method of use of a handheld relaxation device comprises: (a) holding the bolt in a user's hand with the bolt head cradled in the palm of the user's hand and the user's fingertips placed in several of the plurality of bolt head grooves, or the several of the plurality of bolt head notches, or both; (b) holding the cap and the nut in the user's opposite hand with the user's fingertips in several of the plurality of nut grooves, several of the plurality of nut notches, or both; (c) removably attaching the nut to the bolt by threading the nut center onto the bolt threads; (d) removably attaching the cap to the bolt by threading the cap center onto the bolt threads; (e) removably detaching the cap from the bolt by unscrewing the cap center from the bolt threads; and (f) removably detaching the nut from the bolt by unscrewing the nut center from the bolt threads.

Various objects, features, aspects, and advantages of the inventive subject matter will become more apparent from the following detailed description of exemplary embodiments, along with the accompanying figures in which like numerals represent like components.

### BRIEF DESCRIPTION OF THE DRAWINGS

FIG. 1 depicts a perspective view of an exemplary embodiment of a handheld relaxation device.

FIG. 2a depicts a front view of a bolt in an exemplary embodiment a handheld relaxation device.

FIG. 2b depicts a front view of a nut in an exemplary embodiment of a handheld relaxation device.

FIG. 3 depicts a perspective view of a first alternate embodiment of a handheld relaxation device.

FIG. 4 depicts a front view of a first alternate embodiment of a handheld relaxation device.

FIG. 5 depicts a perspective view of a second alternate embodiment of a handheld relaxation device.

FIG. 6 depicts a front view of a second alternate embodiment of a handheld relaxation device.

FIG. 7 depicts a perspective view of a third alternate embodiment of a handheld relaxation device.

FIG. 8 depicts a front view of a third alternate embodiment of a handheld relaxation device.

FIG. 9 depicts a perspective view of a fourth alternate embodiment of a handheld relaxation device.

FIG. 10a depicts a front view of a bolt in a fourth alternate embodiment of a handheld relaxation device.

FIG. 10b depicts a front view of a nut in a fourth alternate embodiment of a handheld relaxation device.

FIG. 10c depicts a front view of a cap in a fourth alternate embodiment of a handheld relaxation device.

### DETAILED DESCRIPTION OF THE INVENTION

The following description is not an admission that any of the information provided herein is prior art or relevant to the present invention, or that any publication specifically or implicitly referenced is prior art. Any publications cited in this description are incorporated by reference herein. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “into” and “on” unless the context clearly dictates otherwise.

As used herein, the term “about” in conjunction with a numeral refers to a range of that numeral starting from 10% below the absolute value of the numeral to 10% above the absolute value of the numeral, inclusive.

As used herein, the terms “handheld relaxation device” and “device” are used interchangeably throughout the disclosure.

The present invention is directed to a handheld relaxation device configured to self-regulate stress and anxiety in individuals. The device is configured to expel nervous energy, relieve stress, and increase blood circulation by generating a rotational movement with the user's hands. In another embodiment, the device is used as a meditation tool. In yet another embodiment, the device is configured to strengthen hand muscles and prevent “computer claw.” The handheld relaxation device can be used in combination with lo-fi music to increase stimulation while using the device. The device is constructed in different sizes, and is configured to fit in the palm of the user's hand. In particular, the device can be effective in promoting focus and concentration in persons with anxiety, ADHD, ADD, hyperactivity, ASD, or those who experience concentration issues.

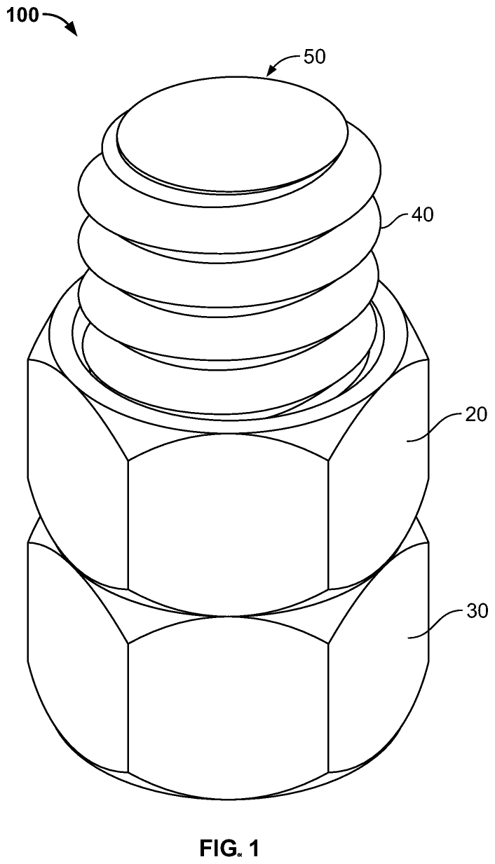

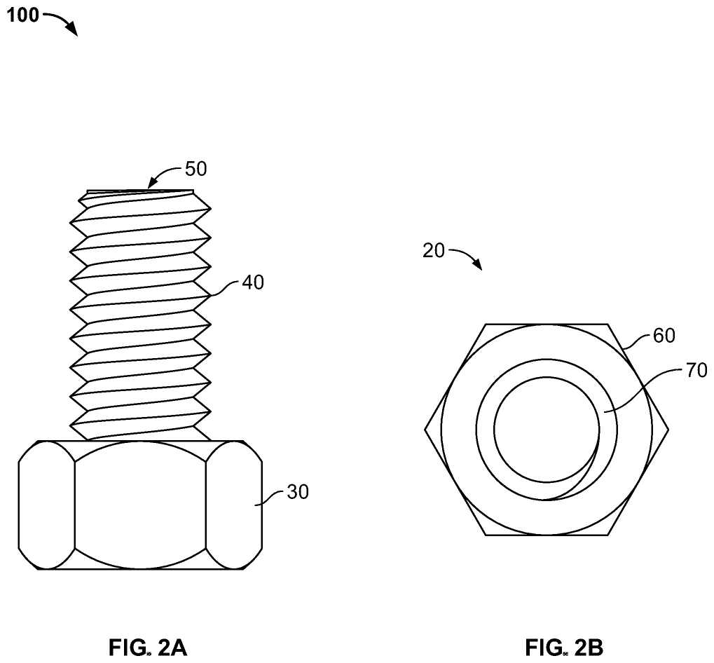

Exemplary configurations of the present invention are depicted in FIGS. 1 to 10, in which handheld relaxation device 100 is configured to be used as an anxiety and/or stress relief device. In one embodiment, handheld relaxation device 100 comprises bolt 50 and nut 20; wherein bolt 50 comprises bolt head 30, and bolt threads 40; and wherein nut 20 comprises nut side 60 and nut center 70 (see FIGS. 1-2). In this embodiment, nut 20 is removably attached to bolt 50 by rotating nut center 70 to engage bolt threads 40 of bolt 50, wherein bolt threads 40 readily engage threads in nut center 70.

In one embodiment, bolt head 30 and nut 20 are in the shape of a hexagon. In this embodiment, bolt head 30 and nut 20 are of equal size.

In an exemplary embodiment, bolt 50 is about 3.44″ in width, about 3.44″ in length, and about 6.25″ in height, and nut 20 is about 3.44″ in width and about 3.44″ in length. In an alternate embodiment, bolt 50 is about 0.67″ in width, about 0.67″ in length, and about 1.51″ in height, and nut 20 is about 0.67″ in width and about 0.67″ in length. In yet another embodiment, bolt 50 is about 2.91″ in width, about 2.91″ in length, and about 4.53″ in height, and nut 20 is about 2.91″ in width and about 2.91″ in length. In yet another embodiment, bolt 50 is about 2.30″ in width, about 2.30″ in length, and about 2.96″ in height, and nut 20 is about 2.30″ in width and about 2.30″ in length.

A method for releasing stress comprises the following steps. Step 1 comprises a user holding bolt 50 in one hand with bolt head 30 cradled in the palm of the user's hand. Step 2 comprises a user holding nut 20 in the opposite hand. Step 3 comprises user turning on lo-fi music using a radio, website, smartphone or other similar device. Step 4 comprises removably attaching nut 20 to bolt 50 by threading nut 20 onto bolt threads 40. Step 5 comprises removably detaching nut 20 from bolt 50 by unscrewing nut 20 from bolt threads 40. Step 6 comprises repeating steps 1-5 until user is in a relaxed, less anxious, and/or preferred state of mind.

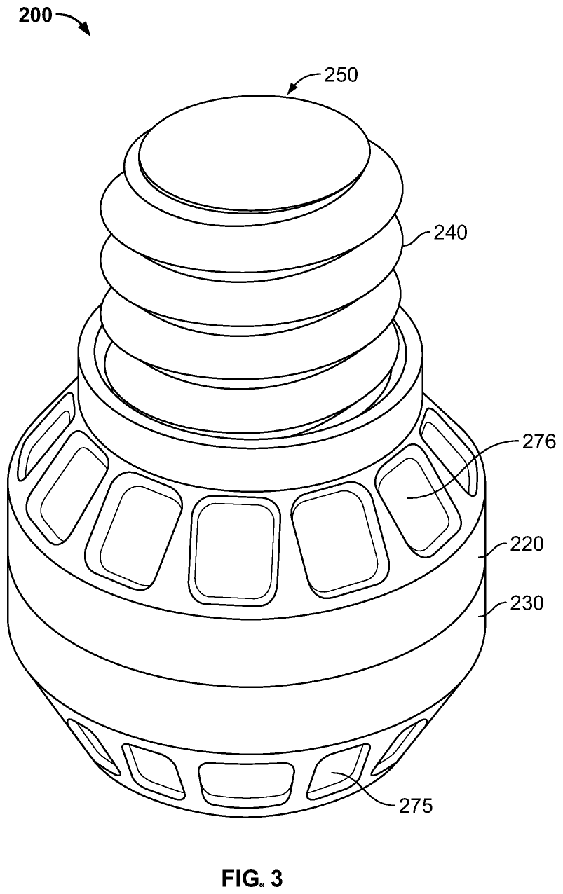

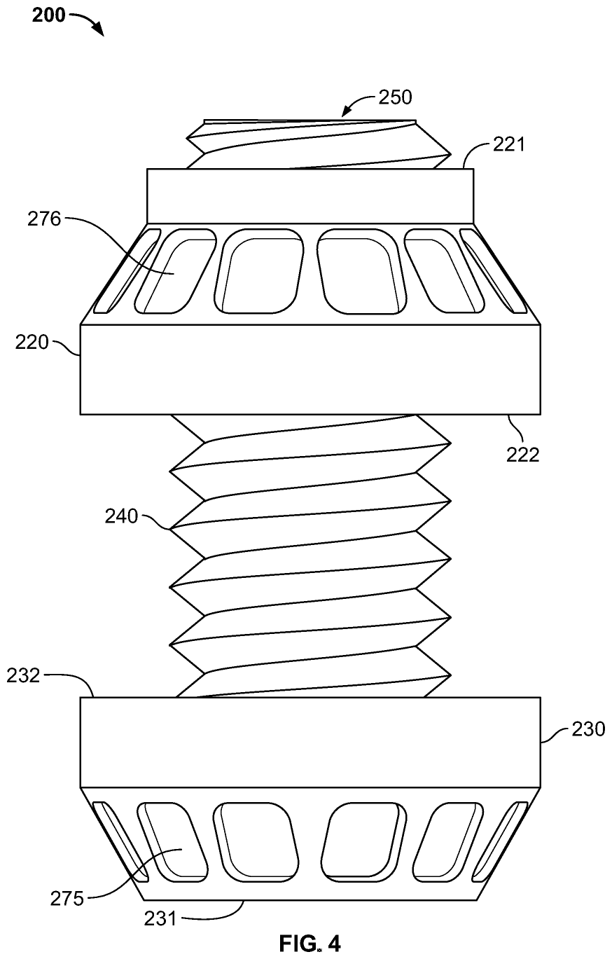

FIGS. 3 to 4 depict a first alternate embodiment of a handheld relaxation device. Handheld relaxation device 200 comprises bolt 250; bolt head 230; wherein bolt head 230 comprises bolt head grooves 275, bolt head top 231, and bold head bottom 232; nut 220, wherein nut 220 comprises nut top 221, nut bottom 222, and nut grooves 276; and bolt threads 240.

In this embodiment, the circumference of bolt head top 231 is smaller in circumference than bolt head bottom 232 and bolt head bottom 232 tapers down to bolt head top 231. Likewise, nut top 221 is smaller in circumference than nut bottom 222 and nut bottom 222 tapers down to nut top 231. Bolt head 230 comprises plurality of bolt head grooves 275 enabling a user to hold bolt head 230 with his/her hand by placing the fingertips in several of the plurality of bolt head grooves 275. Nut 220 comprises plurality of nut grooves 276 enabling a user to hold nut 220 with his/her hand by placing the fingertips in several of the plurality of nut grooves 276. In an alternate embodiment, the circumference of bolt head top 231 is larger than bolt head bottom 232.

In one embodiment, bolt 250 is about 3.44″ in width, about 3.44″ in length, and about 6.25″ in height, and nut 220 is about 3.44″ in width and about 3.44″ in length. In another embodiment, bolt 250 is about 1.34″ in width, about 1.34″ in length, and about 3.03″ in height, and nut 220 is about 1.34″ in width and about 1.34″ in length. In yet another embodiment, bolt 250 is about 1.76″ in width, about 2.24″ in length, and about 3.14″ in height, and nut 220 is about 1.76″ in width and about 2.24″ in length. In another embodiment, bolt 250 is about 2.27″ in width, about 2.27″ in length, and about 3.14″ in height, and nut 220 is about 2.27″ in width and about 2.27″ in length.

An alternate method for releasing stress comprises the following steps. Step 1 comprises a user holding bolt 250 in one hand with bolt head 230 in the palm of the hand and fingertips placed in several of the plurality of bolt head grooves 275. Step 2 comprises a user holding nut 220 in opposite hand with fingertips in several of the plurality of nut grooves 276. Step 3 comprises a user turning on lo-fi music using a radio, website, smartphone, or other similar device. Step 4 comprises a user removably attaching nut 220 to bolt 250 by threading nut 220 onto bolt threads 240. Step 5 comprises removably detaching nut 220 from bolt 250 by unscrewing nut 220 from bolt threads 240. Step 6 comprises repeating steps 1-5 until user is in a relaxed, less anxious, and/or preferred state of mind.

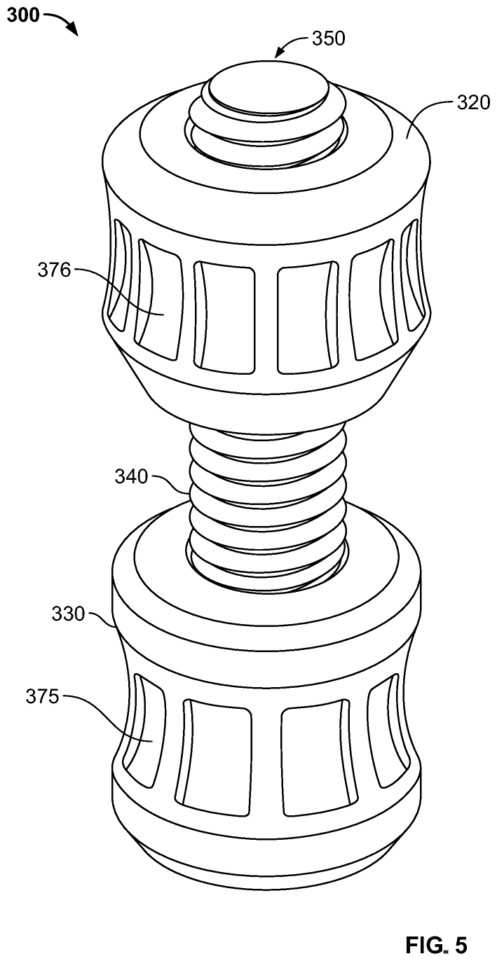

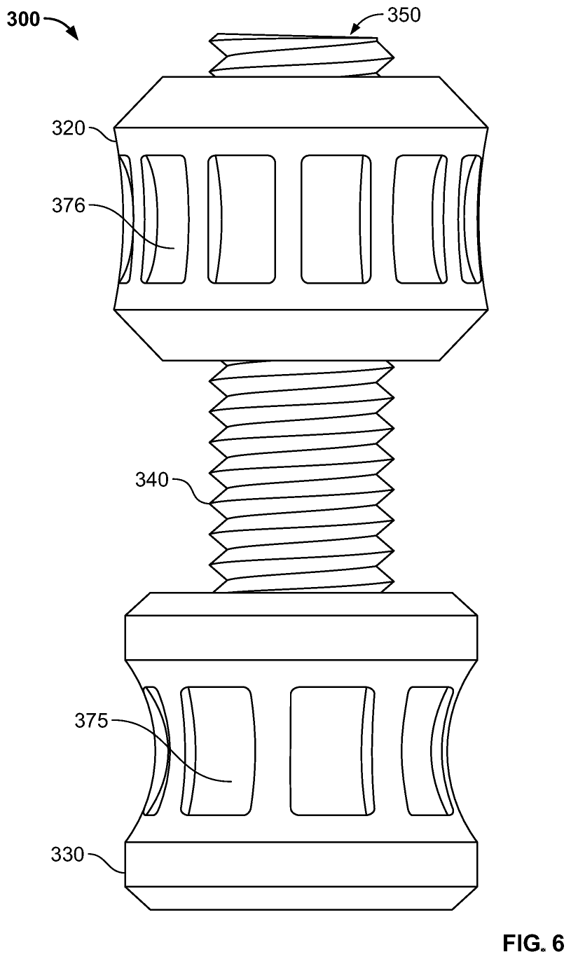

FIGS. 5 to 6 depict a second alternate embodiment of a handheld relaxation device. Handheld relaxation device 300 comprises bolt 350; bolt head 330; wherein bolt head 330 comprises bolt head top 331, bolt head bottom 332, and a plurality of bolt head grooves 375; nut 320, wherein nut 320 comprises nut top 321, nut bottom 322, and a plurality of nut grooves 376; and bolt threads 340.

In this embodiment, bolt head 330 comprises plurality of bolt head grooves 375 enabling a user to hold bolt head 330 with his/her hand by placing their fingertips in several of the plurality of bolt head grooves 375. Nut 320 comprises a plurality of nut grooves 376 enabling a user to hold nut 320 with his/her hand by placing the fingertips in several of the plurality of nut grooves 376.

In an embodiment, the circumference of bolt head top 331 is equal to the circumference of bolt head bottom 332 and bolt head bottom 332 and bolt head top 331 taper towards the middle of bolt head 330. Likewise, the circumference of nut top 321 is equal to the circumference of nut bottom 322 and nut bottom 322 and nut top 321 taper toward the middle of nut 320.

In one embodiment, bolt 350 is about 3.44″ in width, about 3.44″ in length, and about 6.25″ in height, and nut 320 is about 3.44″ in width and about 3.44″ in length. In another embodiment, bolt 350 is about 1.91″ in width, about 2.01″ in length, and about 4.60″ in height, and nut 320 is about 1.91″ in width and about 2.01″ in length. In yet another embodiment, bolt 350 is about 2.91″ in width, about 2.91″ in length, and about 4.53″ in height, and nut 320 is about 2.91″ in width and about 2.91″ in length.

An alternate method for releasing stress comprises the following steps. Step 1 comprises a user holding bolt 350 in one hand with bolt head 330 cradled in the palm of the hand with fingertips placed in several of the plurality of bolt head grooves 375. Step 2 comprises a user holding nut 320 in opposite hand with fingertips in several of the plurality of nut grooves 376. Step 3 comprises a user turning on lo-fi music using a radio, website, smartphone, or other similar device. Step 4 comprises removably attaching nut 320 to bolt 350 by threading nut 320 onto bolt threads 340. Step 5 comprises removably detaching nut 320 from bolt 350 by unscrewing nut 320 from bolt threads 340. Step 6 comprises repeating steps 1-5 until user is in a relaxed, less anxious, and/or preferred state of mind.

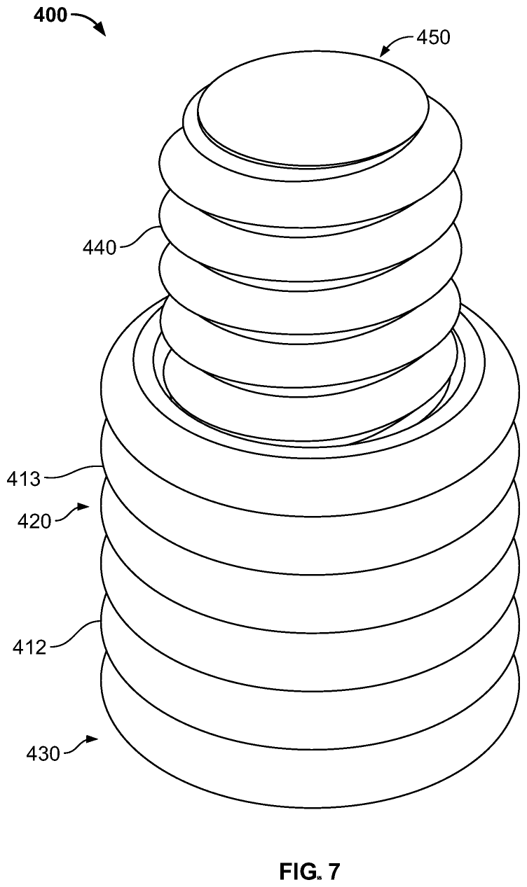

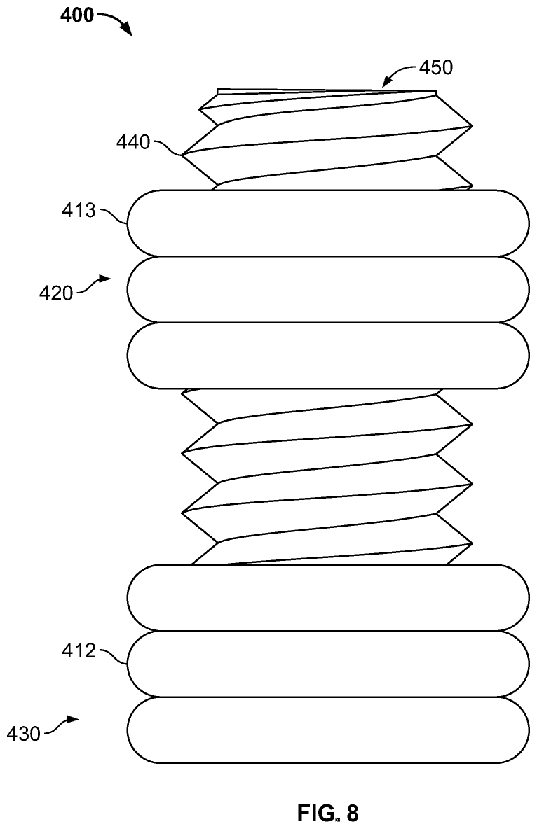

FIGS. 7 to 8 depict a third alternate embodiment of a handheld relaxation device. Handheld relaxation device 400 comprises bolt 450; bolt head 430; nut 420; and bolt threads 440. In this embodiment, bolt head 430 comprises plurality of bolt head swirls 412 enabling a user to hold bolt head 430 with his/her hand by placing the fingertips on plurality of bolt head swirls 412. Nut 420 comprises plurality of nut swirls 413 enabling a user to hold nut 420 with his/her hand by placing the fingertips on several of the plurality of nut swirls 413.

In one embodiment, bolt 450 is about 3.44″ in width, about 3.44″ in length, and about 6.25″ in height, and nut 420 is about 3.44″ in width and about 3.44″ in length. In another embodiment, bolt 450 is about 0.67″ in width, about 0.67″ in length, and about 1.51″ in height, and nut 420 is about 0.67″ in width and about 0.67″ in length. In yet another embodiment, bolt 450 is about 2.91″ in width, about 2.91″ in length, and about 4.53″ in height, and nut 420 is about 2.91″ in width and about 2.91″ in length.

An alternate method for releasing stress comprises the following steps. Step 1 comprises a user holding bolt 450 in one hand with bolt head 430 cradled in the palm of the hand. Step 2 comprises a user holding nut 420 in opposite hand. Step 3 comprises a user turning on lo-fi music using a radio, website, smartphone, or other similar device. Step 4 comprises removably attaching nut 420 to bolt 450 by threading nut 420 onto bolt threads 440. Step 5 comprises removably detaching nut 420 from bolt 450 by unscrewing nut 420 from bolt threads 440. Step 6 comprises repeating steps 1-5 until user is in a relaxed, less anxious, and/or preferred state of mind.

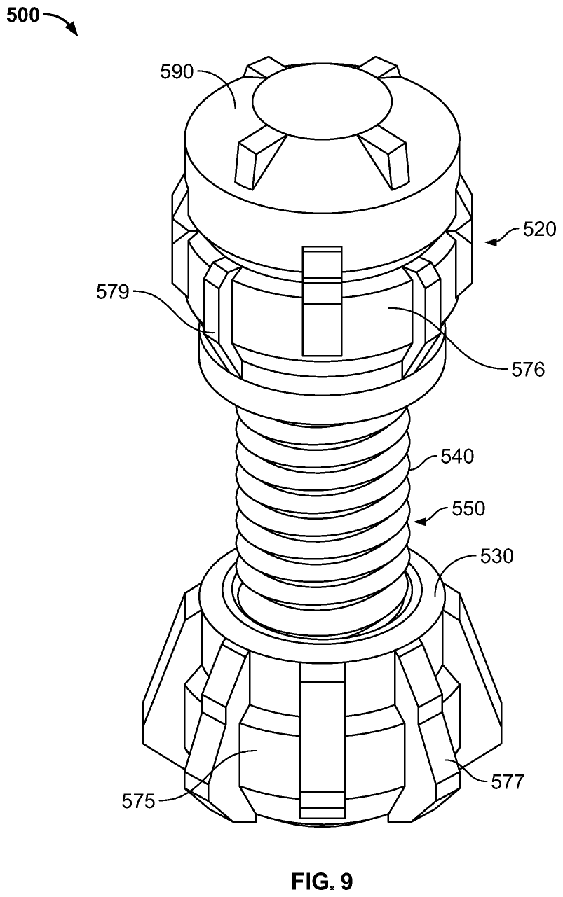

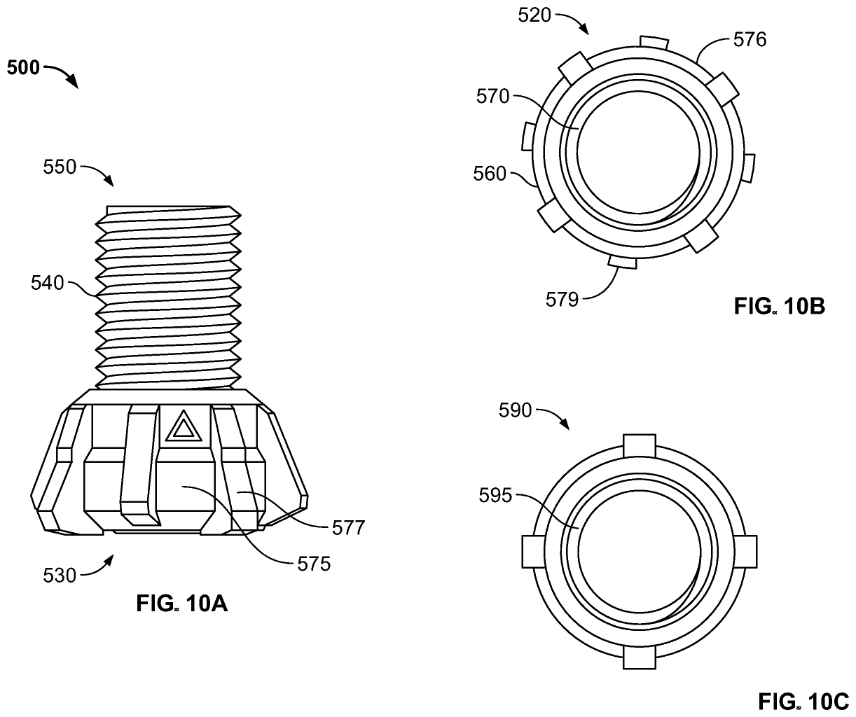

FIGS. 9 to 10 depict a fourth alternate embodiment of a handheld relaxation device. Handheld relaxation device 500 comprises bolt 550; bolt head 530; wherein bolt head 530 comprises plurality of bolt head grooves 575, and plurality of bolt notches 577; bolt threads 540; nut 520, wherein nut 520 comprises nut side 560, nut center 570, plurality of nut grooves 576 and plurality of nut notches 579; and cap 590, wherein cap 590 comprises cap center 595 and cap side 596.

In this embodiment, bolt head 530 comprises plurality of bolt grooves 575 enabling a user to hold bolt head 530 with his/her hand by placing his/her fingertips in several of the plurality of bolt grooves 575. In addition, bolt head 530 comprises plurality of bolt notches 577 enabling a user to hold bolt head 530 with his/her hand by placing his/her fingertips on several of the plurality of bolt notches 577. Likewise, nut 520 comprises plurality of nut grooves 576 enabling a user to hold nut 520 with his/her hand by placing his/her fingertips in several of the plurality of nut grooves 576. In addition, nut 520 comprises plurality of nut notches 579 enabling a user to hold nut 520 with his/her hand by placing his/her fingertips on several of the plurality of nut notches 579.

In an embodiment, nut 520 and cap 590 are removably attached to bolt 550 by sliding nut center 570 and/or cap center 595 over bolt 550, wherein bolt threads 540 readily accept threads in nut center 570 and threads in cap center 595.

In one embodiment, bolt 550 is about 3.44″ in width, about 3.44″ in length, and about 6.25″ in height, nut 520 is about 3.44″ in width and about 3.44″ in length, and cap 590 is about 3.44″ in width and 3.44″ in length. In yet another embodiment, bolt 550 is about 2.91″ in width, about 2.91″ in length, and about 4.53″ in height, nut 520 is about 2.91″ in width and about 2.91″ in length, and cap 590 is about 2.91″ in width and 2.91″ in length.

An alternate method for releasing stress comprises the following steps. Step 1 comprises a user holding bolt 550 in one hand with bolt head 530 cradled in the palm of the hand with fingertips placed in several of the plurality of bolt grooves 575. Step 2 comprises a user holding cap 590 and nut 520 in opposite hand with fingertips in some of the plurality of nut grooves 576. Step 3 comprises a user turning on and listening to lo-fi music using a radio, website, smartphone, or other similar device. Step 4 comprises removably attaching nut 520 to bolt 550 by threading nut 520 onto bolt threads 540. Step 5 comprises removably attaching cap 590 to bolt 550 by threading cap center 595 onto bolt threads 540. Step 6 comprises removably detaching cap 590 from bolt 550 by unscrewing cap 590 from bolt threads 540. Step 7 comprises removably detaching nut 520 from bolt 550 by unscrewing nut 520 from bolt threads 540. Step 8 comprises repeating steps 1-7 until a user is in a relaxed, less anxious, and/or preferred state of mind.

Another alternate method for releasing stress comprises the following steps. Step 1 comprises a user holding bolt 550 in one hand with bolt head 530 cradled in the palm of the hand with fingertips placed in several of the plurality of bolt notches 577. Step 2 comprises a user holding cap 590 and nut 520 in opposite hand with fingertips in nut notches 579. Step 3 comprises a user turning on lo-fi music using a radio, website, smartphone, or other similar device. Step 4 comprises removably attaching nut 520 to bolt 550 by threading nut 520 onto bolt threads 540. Step 5 comprises removably attaching cap 590 to bolt 550 by threading cap center 595 onto bolt threads 540. Step 6 comprises removably detaching cap 590 from bolt 550 by unscrewing cap center 595 from bolt threads 540. Step 7 comprises removably detaching nut 520 from bolt 550 by unscrewing nut 520 from bolt threads 540. Step 8 comprises repeating steps 1-7 until a user is in a relaxed, less anxious, and/or preferred state of mind.

In an exemplary embodiment, material of construction for handheld relaxation device comprises polylactic acid (PLA). In an alternate embodiment, material of construction for handheld relaxation device comprises acrylonitrile butadiene styrene (ABS), nylon, glycol modified polyethylene terephthalate (PETG), polycarbonate, polyvinyl alcohol (PVA), acrylic styrene acrylonitrile (ASA), or any combination thereof. One of ordinary skill in the art can envision other types of material of construction that are within the scope of the present invention.

In an exemplary embodiment, the method of manufacture for handheld relaxation device is three-dimensional (3D) printing using a 3D printer. In an alternate embodiment, the method of manufacture for handheld relaxation device is injection molding.

Thus, specific embodiments of handheld relaxation device and methods of use have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced

1. A handheld relaxation device, comprising:
(a) a bolt comprising a bolt head and bolt threads; and
(b) a nut comprising a plurality of nut side and a nut center, wherein said nut is removably attached to said bolt by rotating said nut center to engage said bolt threads of said bolt; and wherein said bolt threads readily engage threads in said nut center.

2. The handheld relaxation device as recited in claim 1, wherein material of construction for said bolt and said nut comprise acrylonitrile butadiene styrene (ABS), nylon, glycol modified polyethylene terephthalate (PETG), polycarbonate, polyvinyl alcohol (PVA), acrylic styrene acrylonitrile (ASA), or any combination thereof.

3. The handheld relaxation device as recited in claim 1, wherein the method of manufacture for said device comprises 3D printing or injection molding.

4. The handheld relaxation device as recited in claim 1, wherein said bolt head and said nut are in the shape of a hexagon.

5. The handheld relaxation device as recited in claim 1, wherein said bolt head further comprises a plurality of bolt head grooves, a plurality of bolt head notches, bolt head top and a bolt head bottom.

6. The handheld relaxation device as recited in claim 1, wherein said nut further comprises a plurality of nut grooves, plurality of nut notches, a nut top, and a nut bottom.

7. The handheld relaxation device as recited in claim 6, wherein:
i. the circumference of said bolt head top is smaller than the circumference of said bolt head bottom:
ii. said bolt head bottom tapers down to said bolt head top;
iii. the circumference of said nut top is smaller than the circumference of said nut bottom, and
iv. said nut bottom tapers down to said nut top.

8. The handheld relaxation device as recited in claim 5, wherein:
i. the circumference of said bolt head top is equal to the circumference of said bolt head bottom;
ii. said bolt head bottom and said bolt head top taper towards the middle of said bolt head;
iii. the circumference of said nut top is equal to the circumference of said nut bottom, and
iv. said nut bottom and said nut top taper towards the middle of said nut.

9. The handheld relaxation device as recited in claim 1, wherein said bolt head and said nut comprise a plurality of swirls.

10. The handheld relaxation device as recited in claim 1, wherein said bolt head and said nut are of equal size.

11. The handheld relaxation device as recited in claim 1, wherein said bolt is about 0.67″ to 3.44″ in width, about 0.67″ to 3.44″ in length, and about 1.51″ to 6.25″ in height, and said nut is about 0.67″ to 3.44″ in width and about 0.67″ to 3.44″ in length.

12. A handheld relaxation device, comprising:
(a) a bolt comprising a bolt head, wherein bolt head comprises a plurality of bolt grooves and bolt notches and bolt threads;
(b) a nut comprising a plurality of nut side, a nut center, a plurality of nut grooves, and a plurality of nut notches; and
(c) a cap comprising a cap center;
wherein:
i. said nut is removably attached to said bolt by rotating said nut center to engage said bolt threads of said bolt;
ii. said bolt threads readily engage threads in said nut center;
iii. said cap is removably attached to said bolt by rotating said cap center to engage said bolt threads of said bolt; and
iv. said bolt threads readily engage threads in said cap center.

13. The handheld relaxation device as recited in claim 12, wherein said bolt is about 2.91″ to 3.44″ in width, about 2.91″ to 3.44″ in length, and about 4.53″ to 6.25″ in height; said nut is about 2.91″ to 3.44″ in width and about 2.91″ to 3.44″ in length; and said cap is about 2.91″ to 3.44″ in width and is about 2.91″ to 3.44″ in length.

14. The handheld relaxation device as recited in claim 12, wherein material of construction for said bolt and said nut comprise acrylonitrile butadiene styrene (ABS), nylon, glycol modified polyethylene terephthalate (PETG), polycarbonate, polyvinyl alcohol (PVA), acrylic styrene acrylonitrile (ASA), or any combination thereof.

15. The handheld relaxation device as recited in claim 12, wherein the method of manufacture for said device comprises 3D printing or injection molding.

16. A method of using said handheld relaxation device according to claim 6, comprising:
(a) holding said bolt in a user's hand with bolt head cradled in the palm of the hand and fingertips placed in several of said plurality of bolt head grooves;
(b) holding said nut in said user's opposite hand with said users' fingertips in several of said plurality of nut grooves;
(c) removably attaching said nut to said bolt by threading said nut center onto said bolt threads; and
(d) removably detaching said nut from said bolt by unscrewing said nut from said bolt threads.

17. A method of using said handheld relaxation device according to claim 12, comprising:
(a) holding said bolt in a user's hand with said bolt head cradled in the palm of said user's hand and said user's fingertips placed in several of said plurality of bolt head grooves, or said several of said plurality of bolt head notches, or both;
(b) holding said cap and said nut in said user's opposite hand with said user's fingertips in several of said plurality of nut grooves, several of said plurality of nut notches, or both;
(c) removably attaching said nut to said bolt by threading said nut center onto said bolt threads;
(d) removably attaching said cap to said bolt by threading said cap center onto said bolt threads;
(e) removably detaching said cap from said bolt by unscrewing said cap center from said bolt threads; and
(f) removably detaching said nut from said bolt by unscrewing said nut center from said bolt threads.
